(19) United States
(12) Patent Application Publication
(10) Pub. No.: US 2025/0332585 A1
(43) Pub. Date: Oct. 30, 2025

(54) Container for processing a fluidic sample and method for processing a fluidic sample

(71) Applicant: BiopSense Oy, Jyväskylä (FI)

(72) Inventors: Kiia Hartikainen, Jyväskylä (FI); Jouni Toivola, Jyväskylä (FI); Petri Kallberg, Jyväskylä (FI); Veli-Mikko Puupponen, Jyväskylä (FI)

(21) Appl. No.: 19/194,177

(22) Filed: Apr. 30, 2025

(30) Foreign Application Priority Data
Apr. 30, 2024 (FI) 20245543

(51) Int. Cl. B01L 3/00; C12Q 1/6806

(52) U.S. Cl. CPC B01L 3/502; C12Q 1/6806; B01L 2200/0684; B01L 2200/16; B01L 2300/08; B01L 2300/087; B01L 2300/12; B01L 2400/0487

(57) ABSTRACT

According to an example aspect of the present invention, there is provided a container for processing a fluidic sample comprising a first and a second compartment, wherein the second compartment is located above the first compartment and comprises an air permeating vent, which is preferably located at the top of the container; wherein the first compartment comprises an orifice or valve, which is located at the bottom of the container, wherein said orifice or valve is an inlet and an outlet for the fluidic sample.

### CROSS-REFERENCE TO RELATED APPLICATIONS

This application claims the priority of Finnish Patent Application no. 20245543, filed Apr. 30, 2024, which is incorporated by reference herein in its entirety.

### FIELD

The present invention is related to the field of liquid biopsy diagnostics, such as cancer diagnosis. In particular, this invention relates to the use of magnetic beads, and to methods of separating, mixing and concentrating magnetic beads efficiently with a fluid in a closed system. The invention further provides a cartridge and an automated system suitable for detection of circulating tumor-derived small nucleic acids.

### BACKGROUND

It is well-known in the prior art that magnetic particles, such as magnetic beads, can be used as a solid phase to bind a desired target analyte in a biological sample and to separate the bound analyte from the sample utilizing a magnetic field (see, e.g., EP1774341).

For efficient analyte capture, washing and elution, the magnetic particles need to be well dispersed and mixed in the relevant buffers and/or reagents. However, well-known methods for mixing magnetic beads in a liquid media, such as vortexing, can be difficult to automate or to perform in a limited space of an automated cartridge.

In WO2021130415, the present inventors disclosed a method, automated system and cartridge for extraction of cell-free nucleic acids from a blood sample. The present invention provides an improvement to this technology by disclosing a container for processing fluidic samples with magnetic particles which is compatible with the said automated system and cartridge. The present container and its implementation into an automated system provides a solution that overcomes the challenges of the prior art, especially those where magnetic particles are transferred from one container to another for washing and isolation.

### SUMMARY OF THE INVENTION

The invention is defined by the features of the independent claims. Some specific embodiments are defined in the dependent claims.

According to a first aspect of the present invention, there is provided a container for processing a fluidic sample comprising a first and a second compartment, wherein the second compartment is located above the first compartment and comprises an air permeating vent, which is preferably located at the top of the container; wherein the first compartment comprises an orifice or valve, which is located at the bottom of the container, wherein said orifice or valve is an inlet and an outlet for the fluidic sample.

According to a second aspect of the present invention, there is provided a method for processing a fluidic sample using the container of the present invention, the method comprising the steps of:
a) inserting a fluidic sample into the container through the orifice or valve of the first compartment of the container, wherein the fluidic sample has been pre-processed by contacting said fluidic sample with magnetic beads, or wherein said fluidic sample and said magnetic beads are inserted to the container separately in order to obtain a fluidic sample comprising magnetic beads, wherein said magnetic beads can specifically bind to a biological entity of interest possibly present in said fluidic sample;
b) mixing the sample by moving the sample between the first and second compartments,
c) subjecting the mixed sample to a magnetic field within the first compartment to concentrate the magnetic beads against a wall of the first compartment and to hold the magnetic beads on said wall in order to separate the magnetic beads from the rest of the fluidic sample;
d) removing the fluidic part of the sample separated from said magnetic beads in step c) through the orifice or valve of the first compartment in order to produce a concentrated sample of said magnetic beads,
wherein the flow of the fluidic sample in steps a) to d) is controlled by increasing and decreasing the air pressure inside the container through the air vent in the second compartment of said container.

### BRIEF DESCRIPTION OF THE DRAWINGS

FIG. 1 illustrates a longitudinal cross-section of a container comprising two compartments (1, 2) with distinct volumes and a narrow channel (5) between said compartments (1, 2), vent connection (3) at the top of the container and an input/output channel (4) for the liquid sample and liquid reagents. When the container is used as disclosed in the present invention, a sample mixed with magnetic particles is subjected to a magnetic field within the first compartment (1) to concentrate the magnetic beads against a front wall (10) of the first compartment (1) and to hold the magnetic beads on said front wall (10) in order to separate the magnetic beads from the rest of a fluidic sample.

FIG. 2 shows a front view of the container of FIG. 1.

FIG. 3 shows a corner view of the container of FIG. 1.

FIG. 4 illustrates a longitudinal cross-section of a container comprising three compartments (6, 7, 8) with continuous structure, vent connection (3) at the top of the container and an input/output channel (4) for the liquid sample and liquid reagents. Compartment 8 further comprises two separate profiles with different slopes (11, 13) in the back wall. In this embodiment, the magnetic beads can also be held on a front wall (10) by a magnetic field in order to separate the magnetic beads from the rest of a fluidic sample.

FIG. 5 shows a front view of the container of FIG. 4.

FIG. 6 shows a corner view of the container of FIG. 4.

FIG. 7 shows a horizontal cross section projection of the container according to the A-A cutting line as shown in FIG. 4 showing the form of the front wall (10) and the slopes (11, 13) in the back wall seen from above. FIG. 7 shows the bottom of first compartment (8) of the container having a section narrowing towards the orifice with a back wall of a symmetrically stepped transverse section with maximum depth at the centre and areas of intermediate (or minimum) depth (13) between the edges of the back wall and the centre so that the area of minimum depth is located at the edges of the back walls (for instance, there can be a first area of intermediate depth and a second area of intermediate depth at both sides of the centre so that said first area has greater depth than said second area flanking the edges of the back wall). The depth of each slope is measured from the front wall (10). The stepped structure provides improved properties when the liquid sample present in the first compartment is mixed with gas or air bubbles by feeding gas/air as bubbles through the liquid phase from the input/output channel (4) shown in FIG. 4 located in the bottom of said centre part (11) of the stepped section.

FIG. 8 shows detailed views of the container of FIG. 4 depicting the positioning of a magnet (14) onto a front wall (10) demonstrating the relative surface areas and clearance between the magnet and container perimeter.

### EMBODIMENTS

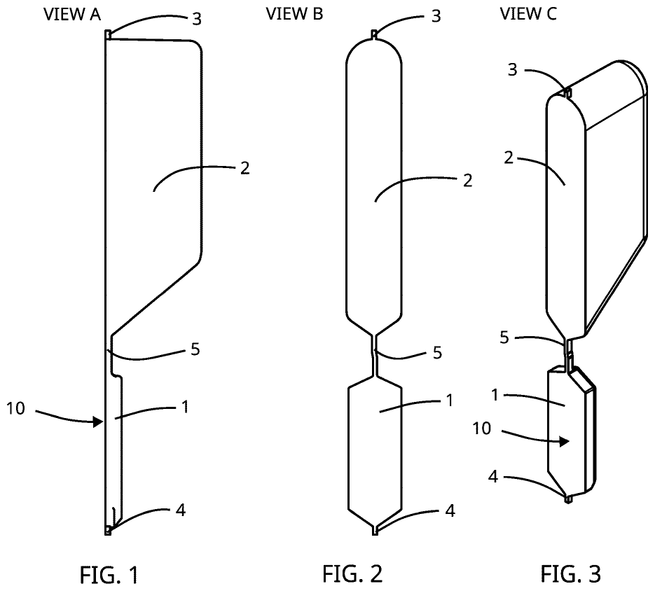

FIGS. 1-3 show a container according to one embodiment of the present invention, which is suitable for isolation of, e.g., nucleic acids from a biological fluid sample by using magnetic particles or for other processes utilizing such particles. In its vertical position, there are flow channels (3, 4) at both ends of the container (i.e. top and bottom). The container comprises two compartments (1, 2) with distinct volumes and a narrow channel (5) between said compartments. This three-part structure renders it possible to achieve both efficient mixing, thermal management, and efficient collection of magnetic particles while moving the sample between the two compartments (1, 2). This structure also prevents the contact and mixing of consecutive buffer and washing reagents in the container during their use in the process such as purification and isolation of nucleic acid from a liquid sample.

These advantages are based on the following features: rapid narrowing of the structure at the bottom of both compartments (1, 2) promotes mixing of the magnetic particles and liquids as the fluid flow rate change due to narrower width of the compartment.

The shallow but wide first compartment (1) of the container supports the collection of magnetic particles, allowing the magnetic field with high flux density close to the surface of the magnet to easily extend through the entire liquid volume, and due to the relatively large cross-section resulting from the extended width of the first compartment (1) the liquid flow is still maintained at a relatively low linear velocity. Similarly, the high surface area to volume ratio of the compartment (1) allows for efficient heating of the liquid media inside the container. In addition, the purity of each consecutive reagents used in the process can be better maintained when the liquid reaches the second compartment (2) in the first step of the process, but at the later stages stays within the first compartment (1), which, due to its smaller volume, can be more efficiently washed compared to the situation where the liquid is constantly in contact with a large surface area in a larger container. The present container also makes it possible to work with a smaller amount of liquid in later stages of the process. Both of these features are important advantages in the cartridge format, where space is limited and reuse of included containers in the different process steps is highly desirable.

When using the container, liquids enter and exit the container through the channel (4) at the bottom of the first compartment (1) and the movement of the fluid is controlled with air pressure fed/released through the vent connection (3) at the top of the container. With this arrangement, the fluid handling inside the container does not require a separate pump, which is an advantage when working with particle suspensions in an automated system. When the liquids are moved inside the container, the air phase is not preferably allowed to mix with the liquid, because this produces difficult-to-remove bubbles that impair the operation of the process. In an embodiment, the introduction of gas bubbles into the liquid is avoided by optically monitoring the surface level of the liquid phase in the container during the steps of the process.

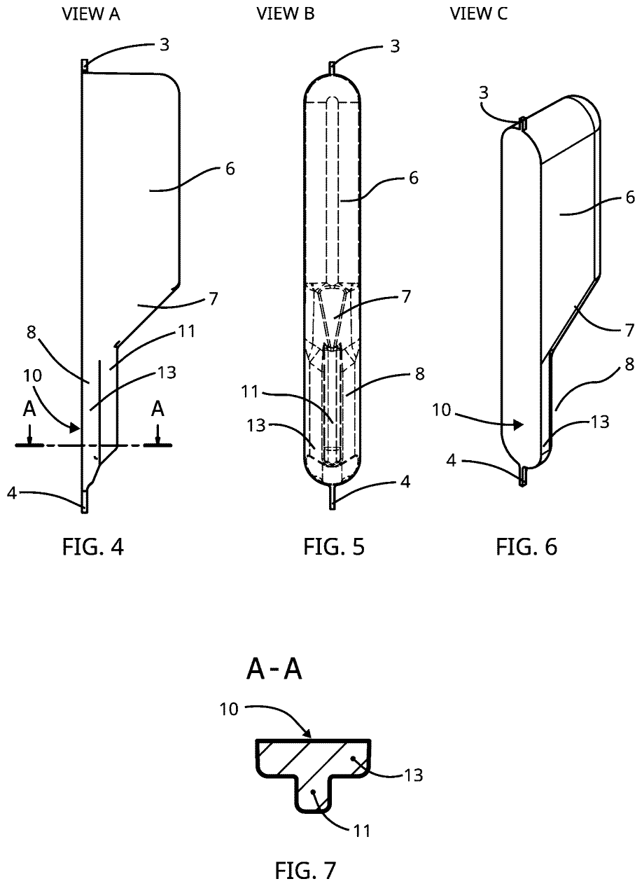

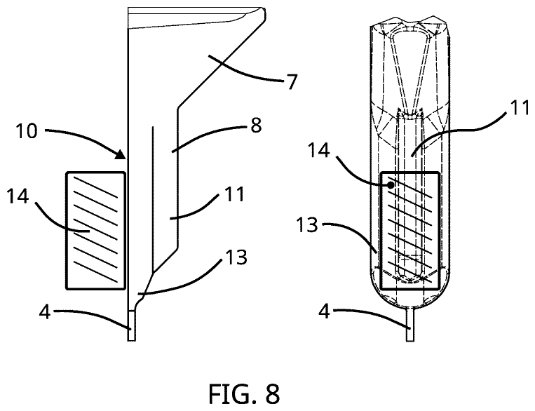

FIGS. 4-7 illustrate a container according to a second embodiment of the present invention, which is suitable for isolation of, e.g., nucleic acids from a biological fluid sample by using magnetic particles or for other processes utilizing such particles. In this second embodiment, the basic principle is similar as in the first embodiment of FIG. 1, i.e., the sample liquid and reagents move through the input/output channel (4) and the air which is used to move the liquids inside the container is fed through the vent channel (3). However, in this embodiment the mixing and collection of magnetic particles take place so that the liquid does not need to be moved between separate compartments through a narrow channel connecting the compartments of the container. In this embodiment, mixing of the sample is further achieved by feeding air as bubbles through the liquid phase from the input/output channel (4). In this solution, the shape of the container has to be more open than in said first embodiment, so that the gas bubbles do not become entrapped in the bottom part of the container (8) and thus create an empty gas-filled void there which would prevent magnetic particles from being collected by a magnet arranged near said bottom part (8) on the front wall (10) outer surface. At the same time, a flat cross section similar to that of the first container (1) in the first embodiment is preferred to reduce the volume. By employing different back wall curvatures (11, 13) at the center and the edge of the container both rapid expansion of the gas along the center line to prevent void formation and a small overall volume to reduce liquid volume have been achieved. As shown in FIG. 8, the said geometry with a wide and shallow area (13) can also accommodate a magnet with a large surface area for efficient particle handling. This configuration prevents the irreversible surface binding of the magnetic particles which takes place when particles are collected onto the front wall (10) inner surface in the very outermost edge areas where the front wall (10) joins with the container body.

As in said first embodiment, the mixing of the liquid in said container is primarily controlled by changing the air supply pressure at different stages of the process. In this way, the best conditions are achieved for both rapid mixing and the collection of particles using said magnet, wherein the slower movement of the magnetic particles enhances the separation process.

It has been found that mixing with air bubbles can be very efficient and the yields of the desired nucleic acids (separated by the magnetic particles) have been even better than in the first embodiment of the present invention.

Introduction of air bubbles also enables the liquid phase to effectively interact with magnetic particles collected onto the wall of the first compartment at vertical height which is above the static level of liquid. This supports further reduction of liquid volume used in later stages during the process.

However, a slight problem in this embodiment is the foaming of the liquid while the air bubbles are added, but this can be reduced with, for example, by adding an anti-foaming agent, such as a silane-based anti-foaming agent, to the liquid sample. It is also possible to reduce this foaming problem by employing controlled surface roughness in the inner walls of the second compartment. In a preferred embodiment, the surface roughness of said inner walls is in the range of Ra 3 to 30 μm.

The present invention is thus directed to a container comprising a first and a second compartment, wherein the second compartment is located above the first compartment and comprises an air permeating vent, which is preferably located at the top of the container; wherein the first compartment comprises an orifice or valve, which is located at the bottom of the container, wherein said orifice or valve is an inlet and an outlet for a fluidic sample.

Preferably, in the first embodiment (as shown in FIG. 3) the total height of said container is about 115 mm so that the height of the first compartment is about 37 mm and the height of the second compartment is about 75 mm.

In a more preferred embodiment, volume of the second compartment (2) is 15-25 ml, preferably 19 ml. The dimensions of said second compartment (2) are 75 mm in height, 25 mm in depth, 13 mm in width.

In a more preferred embodiment, volume of the first compartment (1) is 1-4 ml, preferably 2 ml. The dimensions of said first compartment (1) are 37 mm in height, 4 mm in depth, 13 mm in width.

Preferably, in the second embodiment (as shown in FIG. 6) comprising three compartments (6, 7, 8), the total height of said container is about 100 mm, depth is 20 mm in average and width is 14 mm. Volume is about 20 ml.

In a more preferred embodiment, volume of the second compartment (6) is 15-18 ml, preferably 15 ml. The dimensions of said second compartment (6) are 50 mm in height, 23 mm in depth, and 14 mm in width.

In a more preferred embodiment, total volume of the first compartment (8) is less than 3 ml, preferably 1.5 ml. The overall dimensions of said first compartment (8) are 33 mm in height, 1-9 mm in depth, and 1-14 mm in width. The ratio of the depths for the different curvatures (11,13) is 0:1-1:1 respectively.

In a more preferred embodiment, volume of the third compartment (7) between said first (6) and second (8) compartments is 1-4 ml, preferably 3 ml. The dimensions of said third compartment (7) are 16 mm in height, 8-23 mm in depth, and 14 mm in width.

In a preferred embodiment, the first and the second compartments (1, 2) are interconnected via a channel (5).

In another preferred embodiment, the second compartment (2) has a substantially square shaped or wedge-shaped longitudinal cross-section and the first compartment (1) has a substantially rectangular longitudinal cross-section (seen from the side of the container), see FIG. 1.

In another preferred embodiment, the longitudinal axis of the first compartment (1) is parallel to but spaced apart from the longitudinal axis of the second compartment (2) (seen from the side of the container, see FIG. 1).

In another preferred embodiment, the volume of the second compartment (2) is substantially bigger than the volume of the first compartment (1).

In another preferred embodiment, the ratio of the volumes of the second (2) and first (1) compartments is at least 3:1, preferably at least 5:1.

In another preferred embodiment, the first compartment (8) has a wedge-shaped longitudinal section widening towards the second compartment (6) and narrowing towards said orifice or valve (4) located at the bottom of the container, wherein, preferably, a third wedge-shape compartment (7) is between said first (6) and second (8) compartments, see FIG. 4. In compartment (8), the surfaces (11,13) follow different surface profiles, see FIG. 7.

In another preferred embodiment, the volume of the second compartment (6) is bigger than the volume of the first compartment (8). More preferably, the ratio of the volumes of the second (6) and first (8) compartments is at least 3:1, preferably at least 5:1.

In another preferred embodiment, said air-permeating vent, orifice or valve (3) is connected to a pump. More preferably, the pump allows the movement of the fluidic sample between said first and second compartments via a pressure differential.

In another preferred embodiment, the pump allows the movement of the fluidic sample through said orifice or valve (4) at the bottom of the container.

In another preferred embodiment, said container comprises a transparent wall for optical monitoring of the sample movement inside said container.

The present invention is also directed to a method for processing a fluidic sample using the container of the present invention, the method comprising the steps of:
a) inserting a fluidic sample into the container through the orifice or valve of the first compartment of the container, wherein the fluidic sample has been pre-processed by contacting said fluidic sample with magnetic beads, or wherein said fluidic sample and said magnetic beads are inserted to the container separately in order to obtain a fluidic sample comprising magnetic beads, wherein said magnetic beads can specifically bind to a biological entity of interest possibly present in said fluidic sample;
b) mixing the sample by moving the sample between the first and second compartments, and optionally heating the sample in the first and/or second compartment;
c) subjecting the mixed and optionally heated sample to a magnetic field within the first compartment to concentrate the magnetic beads against a wall of the first compartment and to hold the magnetic beads on said wall in order to separate the magnetic beads from the rest of the fluidic sample; and
d) removing the fluidic part of the sample separated from said magnetic beads in step c) through the orifice or valve of the first compartment in order to produce a concentrated sample of said magnetic beads,
wherein the flow of the fluidic sample in steps a) to d) is controlled by increasing and decreasing the air pressure inside the container through the air vent in the second compartment of said container.

In another preferred embodiment, said method comprise further steps of:
e) inserting a washing buffer to the container through the orifice or valve of the first compartment,
f) removing the washing buffer from said container through the orifice or valve of the first compartment, and
g) removing the concentrated and washed magnetic beads from said container through the orifice or valve of the first compartment, preferably by mixing said concentrated magnetic beads with an elution or a washing buffer.

In another preferred embodiment, in step a) air is passed into the container through the orifice or valve at the bottom of the container to mix the fluidic sample with air bubbles.

In another preferred embodiment, in step a) said magnetic beads are inserted to the container separately as a dry formulation.

In another preferred embodiment, said suspension further comprises an anti-foaming agent.

In another preferred embodiment, the fluidic sample is a plasma sample.

In another preferred embodiment, nucleic acids such as DNA, preferably cfDNA, is/are extracted from the sample onto the surface of the magnetic beads.

The present invention is also directed to a cartridge comprising means for filtering plasma from a blood sample and means for contacting a plasma sample with a binder material specific to nucleic acids, wherein the means for filtering plasma comprises a hollow fiber filter and the means for contacting a plasma sample with a binder material specific to nucleic acids comprises the container of the present invention.

The present invention is further directed to an automated system for extraction of nucleic acid fragments from a blood sample comprising a device with a docking site adapted to receive said cartridge, said device comprising means adapted to operate the blood plasma filtering process in said cartridge and means adapted to operate nucleic acid purification in said cartridge.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e., a singular form, throughout this document does not exclude a plurality.

### EXPERIMENTAL SECTION

### Example 1

Blood plasma was prepared by two-step centrifugation (1900 g, 10 min and 16 000 g, 10 min, both at 21° C.) and then stored at −20° C. short-term or −80° C. long-term. Nucleic acid extraction was performed with carboxylic coated magnetic beads with high DNA binding affinity and a concentration of 50 mg/ml. For both container configurations according to FIGS. 1-3 and 4-6, reagents with the following compositions were used: Proteinase K: 600 mAnsonU/ml, Lysis Buffer: Sodium dodecyl sulfate 40 wt % in water, Binding Buffer: 50 wt % Guanidine thiocyanate and 20 wt % ethanol in water, Washing Buffer 1: 25 wt % Guanidine thiocyanate and 50 wt % ethanol in water, Washing Buffer 2: 80 wt % Ethanol in water, Elution Buffer: 0.1× Tris-EDTA (TE) Buffer, pH 8.4.

For the processing container according to FIGS. 1-3, following sample mixture was prepared: 5 ml plasma, 250 μl Lysis Buffer, 150 μl Magnetic beads and 150 μl of Proteinase K. The sample was heated to 55° C. with a contact heater and cycled between the first and the second container for 10 minutes with an operating pressure of 50 mbar. Then, 6.5 ml of Binding Buffer was introduced into the mixture and the cycling was continued for 10 minutes with a pressure of 650 mbar. A neodymium magnet (12×12×24 mm, grade N45) was then introduced onto the front wall (10) outer surface of the container and cycling at a pressure of 40 mbar was repeated for 3 minutes. The reaction mixture was removed and discarded. The magnet was removed, and 1 ml of Washing Buffer 1 was introduced into the container. The magnetic beads were resuspended into the washing buffer by recycling the solution for 20 seconds at 100 mbar. Then, the bead suspension was transferred into a separate storage container and 3 ml of 50 wt % ethanol was introduced into the processing container. The solution was discarded and 3 ml of 50 wt % ethanol was added again, then discarded. Bead suspension was then transferred back into the processing container, magnet was introduced onto the surface of the container and magnetic beads were collected by cycling at 40 mbar for 30 seconds. The washing buffer was discarded, and 1 ml of Washing Buffer 1 was again introduced into the processing container. Magnet was removed and the beads were resuspended recycling the solution for 20 seconds at 100 mbar. Magnet was reintroduced onto the surface of the container and magnetic beads were collected by cycling at 40 mbar for 30 seconds after which the buffer was discarded. Then, 1 ml of Washing Buffer 2 was introduced into the container and magnetic beads were resuspended by removing the magnet and recycling the solution for 20 seconds at 100 mbar. Magnet was reintroduced onto the container surface and magnetic beads were collected by cycling at 40 mbar for 30 seconds after which the washing buffer was discarded. Another 1 ml of Washing Buffer 2 was introduced into the container and magnet was removed. The magnetic beads were resuspended by recycling the buffer for 20 seconds at 100 mbar.

Suspension was then transferred outside the system and collected into a 2 ml plastic microtube. The magnetic particles were collected onto the side of the tube with a commercial magnetic rack (Bio-Rad Laboratories Inc.) and the supernatant was discarded thereafter. 100 μl of the Elution Buffer was introduced into the tube, magnetic beads were resuspended into the buffer by brief vortexing repeated for 5 times every 1 minutes. The tube was then placed back onto the magnetic rack and the supernatant was collected as the extraction product. For quantitation of DNA, Qubit fluorometer 4 (Thermo Fisher Scientific Inc.) was used. The concentration of the extracted cfDNA was 1.67 ng/ml of plasma (standard deviation (SD) 0.37 ng/ml). Reference yield value from manual extraction with the same reagents was 2.13 ng/ml (SD 0.08 ng/ml).

For the processing container according to FIGS. 4-6, following sample mixture was prepared: 5 ml plasma, 250 μl Lysis Buffer, 150 μl Magnetic beads and 150 μl of Proteinase K. The sample was heated to 55° C. with a contact heater and incubated by feeding air bubbles at a pressure of 40 mbar for 10 minutes. Then, 6.5 ml of Binding Buffer was introduced into the mixture and the bubble mixing was continued for 10 minutes with a pressure of 160 mbar. A neodymium magnet (12×12×24 mm, grade N45) was then introduced onto the front wall (10) outer surface of the container and magnetic beads were collected by bubble mixing at 40 mbar for 3 minutes. The reaction mixture was discarded. Magnet was removed and 1.5 ml of Washing Buffer 1 was introduced into the container. The magnetic beads were resuspended into the washing buffer by bubble mixing for 30 seconds at 100 mbar. Then, the bead suspension was transferred into a separate storage container and 3 ml of 50 wt % ethanol was introduced into the processing container where it was briefly bubble mixed at 550 mbar. The solution was discarded and 3 ml of 50 wt % ethanol was added again, mixed, and then discarded. Bead suspension was then transferred back into the processing container, magnet was introduced onto the surface of the container and magnetic beads were collected by bubble mixing at 40 mbar for 30 seconds. The washing buffer was discarded, and 1 ml of Washing Buffer 1 was again introduced into the processing container. The magnet was removed, and the beads were resuspended by bubble mixing the solution for 30 seconds at 100 mbar. Magnet was reintroduced onto the surface of the container and magnetic beads were collected by bubble mixing at 40 mbar for 30 seconds after which the buffer was discarded. Then, 1 ml of Washing Buffer 2 was introduced into the container and magnetic beads were resuspended by removing the magnet and bubble mixing the solution for 30 seconds at 100 mbar. Magnet was reintroduced onto the container surface and magnetic beads were collected by bubble mixing at 40 mbar for 30 seconds after which the processing container was rinsed with the buffer by applying bubble mixing at 250 mbar to raise the level of bubbles to the high point of the container, after which the buffer was discarded. Another 1 ml of Wash Buffer 2 was introduced into the container and the magnet was removed. The magnetic beads were resuspended by bubble mixing the solution for 30 seconds at 100 mbar.

Suspension was then transferred outside the system and collected into a 2 ml plastic microtube. The manual sample handling was performed exactly as for the container of the first configuration. The concentration of the extracted cfDNA was 2.66 ng/ml of plasma (SD 0.23 ng/ml). Reference yield value from manual extraction with the same reagents was 2.13 ng/ml (SD 0.08 ng/ml).

### CITATION LIST

### Patent Literature

EP1774341
WO2021130415

1. A container for processing a fluidic sample, the container comprising a first and a second compartment,
wherein the second compartment is located above the first compartment and comprises an air permeating vent, which is located at the top of the container,
wherein the first compartment comprises an orifice or valve, which is located at the bottom of the container, wherein said orifice or valve is an inlet and an outlet for the fluidic sample,
wherein the first compartment has a wedge-shaped longitudinal section widening towards the second compartment and narrowing towards said orifice or valve located at the bottom of the container, and
wherein in said first compartment said section narrowing towards said orifice has a back wall with a symmetrically stepped transverse section with maximum depth at the centre and minimum depth at both edges of the back wall.

2. The container according to claim 1, wherein the volume of the second compartment is bigger than the volume of the first compartment.

3. The container according to claim 2, wherein the ratio of the volumes of the second and first compartments is at least 1:3.

4. The container according to claim 1, wherein said air-permeating vent, orifice or valve is connected to a pump.

5. The container according to claim 4, wherein the pump allows the movement of the fluidic sample between said first and second compartments via a pressure differential.

6. The container according to claim 4, wherein the pump allows the movement of the fluidic sample through said orifice or valve at the bottom of the container.

7. The container according to claim 1, wherein said container comprises a transparent wall for optical monitoring of the sample movement inside said container.

8. The container according to claim 1, wherein inner walls of the second compartment comprise areas with surface roughness in the range of Ra 3 to 30 μm.

9. A method for processing a fluidic sample, the method comprising the steps of:
a) inserting a fluidic sample into the container according to claim 1 through the orifice or valve of the first compartment of the container, wherein the fluidic sample has been pre-processed by contacting said fluidic sample with magnetic beads, or wherein said fluidic sample and said magnetic beads are inserted to the container separately in order to obtain a fluidic sample comprising magnetic beads, wherein said magnetic beads can specifically bind to a biological entity of interest possibly present in said fluidic sample;
b) mixing the sample by moving the sample between the first and second compartments, and optionally heating the sample in the first and/or second compartment;
c) subjecting the mixed and optionally heated sample to a magnetic field within the first compartment to concentrate the magnetic beads against a wall of the first compartment and to hold the magnetic beads on said wall in order to separate the magnetic beads from the rest of the fluidic sample; and
d) removing the fluidic part of the sample separated from said magnetic beads in step c) through the orifice or valve of the first compartment in order to produce a concentrated sample of said magnetic beads,
wherein the flow of the fluidic sample in steps a) to d) is controlled by increasing and decreasing the air pressure inside the container through the air vent in the second compartment of said container, and wherein mixing the fluidic sample or washing buffer by feeding into the container through the orifice or valve at the bottom of the container air/gas bubbles is employed in one or more steps of a) to d).

10. The method according to claim 9 comprising further steps of:
e) inserting a washing buffer to the container through the orifice or valve of the first compartment,
f) removing the washing buffer from said container through the orifice or valve of the first compartment, and
g) removing the concentrated and washed magnetic beads from said container through the orifice or valve of the first compartment, preferably by mixing said concentrated magnetic beads with an elution or a washing buffer.

11. The method according to claim 10, wherein in step a) and/or e) air or gas is passed into the container through the orifice or valve at the bottom of the container to mix the fluidic sample or washing buffer with air/gas bubbles.

12. The method according to claim 10, wherein mixing the fluidic sample or washing buffer by feeding into the container through the orifice or valve at the bottom of the container air/gas bubbles is employed in one or more steps of a) to g).

13. The method according to claim 12, wherein the feed of air/gas bubbles is used in i) concentrating the magnetic beads against a wall of the first compartment, ii) removing the magnetic beads from the wall of the first compartment, and/or iii) mixing the sample in any of the washing steps.

14. The method according to claim 11, wherein said suspension further comprises an anti-foaming agent.

15. The method according to claim 9, wherein the fluidic sample is a plasma sample.

16. The method according to claim 15, wherein DNA, preferably cfDNA, is extracted from the sample onto the surface of the magnetic beads.
